(19) United States
(12) Patent Application Publication
(10) Pub. No.: US 2025/0185962 A1
(43) Pub. Date: Jun. 12, 2025

(54) ELECTRONIC DEVICE AND TRAINING METHOD FOR SCREENING ASD AND ASD SYMPTOM SEVERITY BASED ON RETINAL IMAGES

(71) Applicant: UIF (UNIVERSITY INDUSTRY FOUNDATION), YONSEI UNIVERSITY, Seoul (KR)

(72) Inventors: Keun Ah CHEON, Seoul (KR); Yu Rang PARK, Seoul (KR)

(73) Assignee: UIF (UNIVERSITY INDUSTRY FOUNDATION), YONSEI UNIVERSITY, Seoul (KR)

(21) Appl. No.: 18/972,670

(22) Filed: Dec. 6, 2024

(30) Foreign Application Priority Data
Dec. 8, 2023 (KR) 10-2023-0177494
Apr. 17, 2024 (KR) 10-2024-0051727

(51) Int. Cl. A61B 5/16; A61B 5/00; G06T 7/00; G06T 7/10

(52) U.S. Cl. CPC A61B 5/165; A61B 5/7267; G06T 7/0012; G06T 7/10; G06T 2207/20084; G06T 2207/30041

(57) ABSTRACT

Disclosed is an electronic device for screening autism spectrum disorder (ASD) and ASD symptom severity based on a retina image including an input unit that receives the retina image, a classification model that classifies whether there is the ASD, and the ASD symptom severity based on the retina image by using a deep learning algorithm, and at least one processor that controls the input unit and the classification model. The at least one processor is configured to preprocess the received retina image, to train the classification model such that the classification model classifies the ASD and typical development (TD) by using the preprocessed retina image, and classifies the ASD symptom severity, and to allow the trained classification model to screen whether there is the ASD and the ASD symptom severity depending on the input retina image.

### CROSS-REFERENCE TO RELATED APPLICATIONS

A claim for priority under 35 U.S.C. § 119 is made to Korean Patent Application Nos. 10-2023-0177494 filed on Dec. 8, 2023 and 10-2024-0051727 filed on Apr. 17, 2024 in the Korean Intellectual Property Office, the entire contents of which are hereby incorporated by reference.

### BACKGROUND

Embodiments of the present disclosure described herein relate to an electronic device for screening ASD and a training method thereof, and more particularly, relate to an electronic device for screening ASD and ASD symptom severity based on retina images by using an ensemble model utilizing a deep learning algorithm, and a training method thereof.

Autism Spectrum Disorder (ASD) is a category of neurodevelopmental disorders characterized by persistent impairments in reciprocal social communication from early childhood as well as restricted and repetitive patterns of behavior, interests, and the range of activities.

Screening for ASD may only be done by trained professionals who are specially trained to perform the screening, and thus limited resources make it difficult to do so for a large number of children.

In conventional patent documents, neurodevelopmental disorders are easily screened by outputting information on the probability that neurodevelopmental disorders is diagnosed by using retina image data of infants and young children. However, the severity of neurodevelopmental disorders is not screened by simply detecting the part of the image corresponding to the optic nerve papilla.

However, it is essential to determine the severity of ASD, not just the presence of ASD, because the treatment and subsequent training methods vary greatly depending on the severity.

Accordingly, there is a need for a deep learning model capable of performing training in consideration of differences between people with ASD and typical development (TD), and differences between mild ASD and severe ASD, and determining the presence and severity of ASD.

### SUMMARY

Embodiments of the present disclosure provide an electronic device for screening ASD and ASD symptom severity based on retina images by using an ensemble model with a deep learning algorithm, and a training method thereof.

Problems to be solved by the present disclosure are not limited to the problems mentioned above, and other problems not mentioned will be clearly understood by those skilled in the art from the following description.

According to an embodiment, an electronic device for screening autism spectrum disorder (ASD) and ASD symptom severity based on a retina image includes an input unit that receives the retina image, a classification model that classifies whether there is the ASD, and the ASD symptom severity based on the retina image by using a deep learning algorithm, and at least one processor that controls the input unit and the classification model. The at least one processor is configured to preprocess the received retina image, to train the classification model such that the classification model classifies the ASD and typical development (TD) by using the preprocessed retina image, and classifies the ASD symptom severity, and to allow the trained classification model to screen whether there is the ASD and the ASD symptom severity depending on the input retina image.

According to an embodiment, a training method for screening ASD and ASD symptom severity based on a retina image as a training method of an electronic device including an input unit configured to receive a retina image and a classification model using a deep learning algorithm includes preprocessing the received retina image, training the classification model such that the classification model classifies ASD and TD by using the preprocessed retina image, and classifies ASD symptom severity, and allowing the trained classification model to screen whether there is the ASD and the ASD symptom severity depending on the input retina image.

Besides, a computer program stored in a computer-readable recording medium for implementing the present disclosure may be further provided.

In addition, a computer-readable recording medium for recording a computer program for implementing the present disclosure may be further provided.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a block diagram schematically illustrating a configuration of an electronic device for screening ASD and ASD symptom severity based on retina images, according to an embodiment of the present disclosure;

FIG. 2 is a flowchart illustrating a training method of an electronic device for screening ASD and ASD symptom severity based on retina images, according to an embodiment of the present disclosure;

FIG. 3 is a block diagram schematically illustrating a configuration of a classification model of an electronic device for screening ASD and ASD symptom severity based on retina images, according to an embodiment of the present disclosure;

FIG. 4 illustrates participant characteristics constituting learning data of an electronic device for screening ASD and ASD symptom severity based on retina images, according to an embodiment of the present disclosure;

FIG. 5 illustrates a mean performance difference according to a classification model configuration of an electronic device for screening ASD and ASD symptom severity based on retina images, according to an embodiment of the present disclosure;

FIGS. 6A to 6C are graphs showing predictive uncertainty according to a data set of an electronic device for screening ASD and ASD symptom severity based on retina images, according to an embodiment of the present disclosure;

FIGS. 7A and 7B are graphs showing AUROC according to progressively erasing of retina images, which are training data of an electronic device for screening ASD and ASD symptom severity based on retina images, according to an embodiment of the present disclosure; and

FIGS. 8A and 8B are graphs showing AUROC according to progressively erasing of retina images, which are training data of an electronic device for screening ASD and ASD symptom severity based on retina images, according to an embodiment of the present disclosure.

### DETAILED DESCRIPTION

The same reference numerals denote the same elements throughout the present disclosure. The present disclosure does not describe all elements of embodiments. Well-known content or redundant content in which embodiments are the same as one another will be omitted in a technical field to which the present disclosure belongs. A term such as ‘unit, module, member, or block’ used in the specification may be implemented with software or hardware. According to embodiments, a plurality of ‘units, modules, members, or blocks’ may be implemented with one component, or a single ‘unit, module, member, or block’ may include a plurality of components.

Throughout this specification, when it is supposed that a portion is “connected” to another portion, this includes not only a direct connection, but also an indirect connection. The indirect connection includes being connected through a wireless communication network.

Furthermore, when a portion “comprises” a component, it will be understood that it may further include another component, without excluding other components unless specifically stated otherwise.

Throughout this specification, when it is supposed that a member is located on another member “on”, this includes not only the case where one member is in contact with another member but also the case where another member is present between two other members.

Terms such as ‘first’, ‘second’, and the like are used to distinguish one component from another component, and thus the component is not limited by the terms described above.

Unless there are obvious exceptions in the context, a singular form includes a plural form.

In each step, an identification code is used for convenience of description. The identification code does not describe the order of each step. Unless the context clearly states a specific order, each step may be performed differently from the specified order.

Hereinafter, operating principles and embodiments of the present disclosure will be described with reference to the accompanying drawings.

In this specification, an ‘apparatus according to an embodiment of the present disclosure’ includes all various devices capable of providing results to a user by performing arithmetic processing. For example, the apparatus according to an embodiment of the present disclosure may include all of a computer, a server device, and a portable terminal, or may be in any one form.

Here, for example, the computer may include a notebook computer, a desktop computer, a laptop computer, a tablet PC, a slate PC, and the like, which are equipped with a web browser.

The server device may be a server that processes information by communicating with an external device and may include an application server, a computing server, a database server, a file server, a game server, a mail server, a proxy server, and a web server.

For example, the portable terminal may be a wireless communication device that guarantees portability and mobility, and may include all kinds of handheld-based wireless communication devices such as a smartphone, a personal communication system (PCS), a global system for mobile communication (GSM), a personal digital cellular (PDC), a personal handyphone system (PHS), a personal digital assistant (PDA), International Mobile Telecommunication (IMT)-2000, a code division multiple access (CDMA)-2000, W-Code Division Multiple Access (W-CDMA), and Wireless Broadband Internet (WiBro) terminal, and a wearable device such as a timepiece, a ring, a bracelet, an anklet, a necklace, glasses, a contact lens, or a head-mounted device (HMD).

Functions related to artificial intelligence according to an embodiment of the present disclosure are operated through a processor and a memory. The processor may consist of one or more processors. In this case, the one or more processors may be a general-purpose processor (e.g., a CPU, an AP, or a digital signal processor (DSP)), a graphics-dedicated processor (e.g., a GPU or a vision processing unit (VPU)), or an artificial intelligence (AI)-dedicated processor (e.g., an NPU). Under control of the one or more processors, input data may be processed depending on an AI model, or a predefined operating rule stored in the memory. Alternatively, when the one or more processors are AI-dedicated processors, the AI-dedicated processor may be designed with a hardware structure specialized for processing a specific AI model.

The predefined operating rule or the artificial intelligence model is created through learning. Here, being created through learning means creating the predefined operating rule or the artificial intelligence model configured to perform desired features (or purposes) as a basic artificial intelligence model is learned by using pieces of learning data by a learning algorithm. This learning may be performed by an apparatus itself, on which the artificial intelligence according to an embodiment of the present disclosure is performed, or may be performed through a separate server and/or system. For example, the learning algorithm may include supervised learning, unsupervised learning, semi-supervised learning, or reinforcement learning, but may not be limited to the above example.

An artificial intelligence model may be composed of a plurality of neural network layers. The plurality of neural network layers respectively have a plurality of weight values, and each of the plurality of neural network layers performs neural network calculation through calculations between the calculation result of the previous layer and the plurality of weight values. The plurality of weight values of the plurality neural network layers may be optimized by the learning result of the artificial intelligence model. For example, during a learning process, the plurality of weight values may be updated such that a loss value or a cost value obtained from the artificial intelligence model is reduced or minimized. The artificial neural network may include a deep neural network (DNN). The artificial neural network may be, for example, a convolutional neural network (CNN), a deep neural network (DNN), a recurrent neural network (RNN), a restricted Boltzmann machine (RBM), a deep belief network (DBN), a bidirectional recurrent deep neural network (BRDNN), or a deep Q-network, but is not limited to the above-described example.

According to an embodiment of the present disclosure, a processor may implement artificial intelligence. The artificial intelligence may refer to an artificial neural network-based machine learning method that allows a machine to perform learning by simulating human biological neurons. The methodology of artificial intelligence may be classified as supervised learning, in which a solution (output data) to a problem (input data) is determined by providing input data and output data together as training data depending on a learning method, unsupervised learning, in which only input data is provided without output data, and thus the solution (output data) to the problem (input data) is not determined, and reinforcement learning, in which a reward is given from an external environment whenever an action is taken in a current state, and thus learning progresses to maximize this reward. Moreover, the methodology of artificial intelligence may also be categorized depending on architecture, which is the structure of the learning model. The architecture of deep learning technology widely used may be categorized into convolutional neural networks (CNN), recurrent neural networks (RNN), transformers, and generative adversarial networks (GAN).

Each of the apparatus and the system may include an artificial intelligence model. The artificial intelligence model may be a single artificial intelligence model or may be implemented as a plurality of artificial intelligence models. The artificial intelligence model may be composed of neural networks (or artificial neural networks) and may include a statistical learning algorithm that mimics biological neurons in machine learning and cognitive science. The neural network may refer to a model as a whole having the ability to solve problems as artificial neurons (nodes), which form a network by connecting synapses, changes the strength of their synaptic connections through learning. Neurons in the neural network may include the combination of weight values or biases. The neural network may include one or more layers consisting of one or more neurons or nodes. For example, the apparatus may include an input layer, a hidden layer, and an output layer. The neural network constituting the apparatus may infer the result (output) to be predicted from an arbitrary input by changing a weight value of a neuron through learning.

The processor may create a neural network, may train or learn a neural network, or may perform operations based on received input data, and then may generate an information signal or may retrain the neural network based on the performed results. Models of a neural network may include various types of models such as a convolution neural network (CNN) (e.g., GoogleNet, AlexNet, or VGG Network), a region with convolution neural network (R-CNN), a region proposal network (RPN), a recurrent neural network (RNN), a stacking-based deep neural network (S-DNN), a state-space dynamic neural network (S-SDNN), a deconvolution network, a deep belief network (DBN), a restricted Boltzman machine (RBM), a fully convolutional network, a long short-term memory (LSTM) Network, and a classification network, but is not limited thereto. The processor may include one or more processors for performing computations according to the models of the neural network. For example, the neural network may include a deep neural network.

It will be understood by those skilled in the art that a neural network may include any neural network, but is not limited to a convolutional neural network (CNN), a recurrent neural network (RNN), a perceptron, a multilayer perceptron, a feed forward (FF), a radial basis network (RBF), a deep feed forward (DFF), a long short term memory (LSTM), a gated recurrent unit (GRU), an auto encoder (AE), a variational auto encoder (VAE), a denoising auto encoder (DAE), a sparse auto encoder (SAE), a Markov chain (MC), a Hopfield network (HN), a Boltzmann machine (BM), a restricted Boltzmann machine (RBM), a deep belief network (DBN), a deep convolutional network (DCN), a deconvolutional network (DN), a deep convolutional inverse graphics network (DCIGN), a generative adversarial network (GAN), a liquid state machine (LSM), an extreme learning machine (ELM), an echo state network (ESN), a deep residual network (DRN), a differentiable neural computer (DNC), a neural turning machine (NTM), a capsule network (CN), a Kohonen network (KN), and an attention network (AN).

According to an embodiment of the present disclosure, the processor may use various artificial intelligence structures and algorithms such as a convolution neural network (CNN) (e.g., GoogleNet, AlexNet, or VGG Network), a region with convolution neural network (R-CNN), a region proposal network (RPN), a recurrent neural network (RNN), a stacking-based deep neural network (S-DNN), a state-space dynamic neural network (S-SDNN), a deconvolution network, a deep belief network (DBN), a restricted Boltzman machine (RBM), a fully convolutional network, a long short-term memory (LSTM) Network, a classification network, Generative Modeling, eXplainable AI, Continual AI, Representation Learning, AI for Material Design, algorithms for natural language processing (e.g., BERT, SP-BERT, MRC/QA, Text Analysis, Dialog System, GPT-3, and GPT-4), algorithms for vision processing (e.g., Visual Analytics, Visual Understanding, Video Synthesis, and ResNet), algorithms for data intelligence (e.g., Anomaly Detection, Prediction, Time-Series Forecasting, Optimization, Recommendation, and Data Creation), but is not limited thereto. Hereinafter, embodiments of the present disclosure will be described in detail with reference to accompanying drawings.

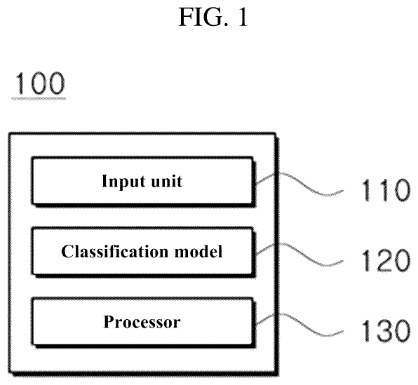

FIG. 1 is a block diagram schematically illustrating a configuration of an electronic device for screening ASD and ASD symptom severity based on retina images, according to an embodiment of the present disclosure. Hereinafter, an electronic device and a training method according to an embodiment of the present disclosure will be described with reference to FIGS. 1 to 8.

ASD patients have no visible biomarkers, thereby making them difficult to determine ASD outwardly. Trained professionals need to determine ASD based on one of the specialized criteria for determining ASD. The present disclosure may determine whether there is ASD, as well as ASD symptom severity, based on structural retina change images, to which brain changes are reflected, by including visual pathway abnormalities through embryonic and anatomical connections.

In other words, retina images of TD and ASD patients, in an embodiment, the sizes of alpha and beta zones in the retina or the ratio of the alpha zone to the beta zone, are different from each other, and the presence of ASD and ASD symptom severity may be screened or analyzed by using the difference.

As illustrated in FIG. 1, an electronic device 100 for screening ASD and ASD symptom severity based on a retina image according to an embodiment of the present disclosure may include an input unit 110 for receiving the retina image, a classification model 120 for classifying ASD and ASD symptom severity based on the retina image by using a deep learning algorithm, and at least one processor 130 for controlling the input unit 110 and the classification model 120.

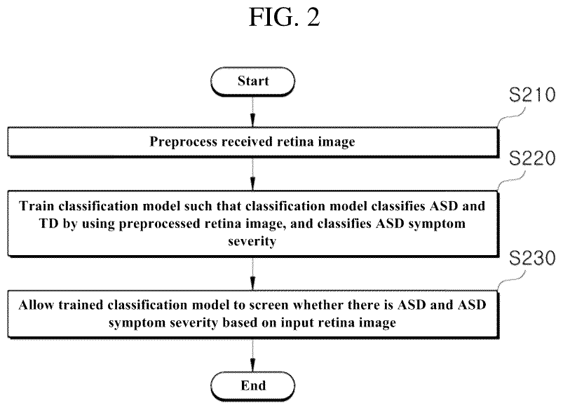

As shown in FIG. 2, the at least one processor 130 of the electronic device 100 according to an embodiment of the present disclosure may perform a training method of preprocessing the received retina image (S210), training the classification model 120 such that the classification model 120 classifies ASD and TD by using the preprocessed retina image and classifies the ASD symptom severity (S220), and screening, by the trained classification model, whether there is ASD and ASD symptom severity based on the input retina image (S230).

The retina image may include information about sizes of an alpha zone and a beta zone, and the ratio of the alpha zone and the beta zone to the entire pupil.

The input unit 110 may receive a retina image directly from a device capturing a retina, or from a memory in which the retina image is stored.

The classification model 120 may include a first single model or a first ensemble model, which determines whether there is ASD, and a second ensemble model that determines ASD symptom severity.

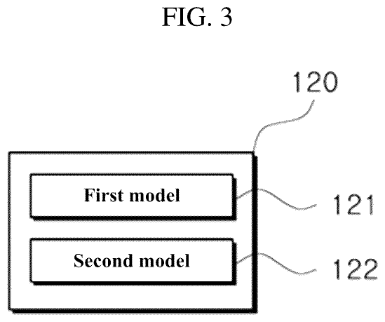

As shown in FIG. 3, the model that determines whether the ASD is present may be defined as a first model 121, and the model that determines the ASD symptom severity may be defined as a second model 122. In the case, the first model 121 and the second model 122 are functionally distinct from each other. For example, one deep learning model may determine whether there is ASD and whether there is ASD symptom severity, or separate deep learning models may determine whether there is ASD and whether there is ASD symptom severity, respectively.

The first model 121 may be either the first single model or the first ensemble model. The first ensemble model is an ensemble model that determines whether there is ASD, and has higher performance and greater ability to quantify predictive uncertainty than the first single model. As the size of a data set increases, the first ensemble model may be used.

Moreover, the second model 122 may be a second ensemble model. In the case of ASD symptom severity, the complexity and predictive uncertainty are higher, and thus utilizing a deep ensemble model is more desirable for prediction efficiency.

Accordingly, the first ensemble model or the second ensemble model may be a deep ensemble-based deep learning model. The deep ensemble-based algorithm may be used to determine both whether there is ASD and whether there is ASD symptom severity, by using the single classification model 120.

Meanwhile, the classification model 120 may be a convolutional neural network that uses ResNeXt-50 network as a backbone. The classification model 120 may be built as a deep ensemble-based deep learning model by using a convolutional neural network.

The at least one processor 130 of the electronic device 100 according to an embodiment of the present disclosure may classify training or verification data of the classification model 120 into ASD and TD depending on only a diagnostic and statistical manual of mental disorders, fifth edition (DSM-5) criteria, and may classify the training or verification data of the classification model into the ASD and the TD depending on the DSM-5 criterion and ADOS-2 score.

In an embodiment, the TD and the diagnosed ASD may be distinguished from each other by using only the first criterion being DSM-5 criterion. The ASD may be screened by distinguishing the diagnosed ASD and the TD by using the first criterion being DSM-5 criterion and a second criterion being ADOS-2 score.

Furthermore, in an embodiment, the training or verification data of the classification model 120 may be classified depending on the ASD symptom severity based on the score calculated by calculating ADOS-2 calibrated severity score and SRS-2 T score. In this case, for example, the ADOS-2 calibrated severity score may be classified into values greater than 8 and less than 8, and the SRS-2 T score may be classified into values greater than 76 and less than 76.

In the meantime, the at least one processor 130 of the electronic device 100 according to an embodiment of the present disclosure may perform random undersampling on a retina image before the ASD and the ASD symptom severity are classified, and may perform data segmentation depending on at least one segmentation ratio.

The data set may be randomly segmented into training data and validation data, and 10-fold cross validation may be performed to obtain generalized results on the performance of a classification model.

The data segmentation is performed at a participant level and may be stratified by result variables. The random undersampling may be performed due to the imbalance according to the ASD symptom severity, and the segmentation ratio may be set in various ways to evaluate the robustness and consistency of the prediction.

For example, performance evaluation may be performed by performing the data segmentation depending on various ratios such as 80:20 and 90:10.

Also, the at least one processor 130 may perform preprocessing of setting and labeling an alpha zone and a beta zone as an ROC area in the retina image before classifying the ASD and the ASD symptom severity.

An area around an optic nerve head in the retina may be split into the alpha zone, the beta zone, and a gamma zone. Among these, the diagnosed ASD and the TD show have differences depending on sizes of the alpha zone and the beta zone and the ratio between the alpha zone and beta zone. The alpha zone and the beta zone may be detected and may be set and labeled as the ROC area.

Accordingly, the classification model 120 may perform training through the process. The trained classification model 120 may output participant-level area under the receiver operating characteristic curve (AUROC), sensitivity, and specificity. 95% CI may be evaluated through a bootstrapping method by using 1000 resamples.

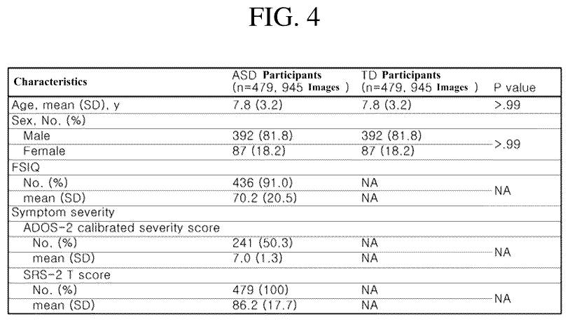

As shown in FIG. 4, the classification model 120 according to an embodiment of the present disclosure performs training and verification by using 1890 eye images of 958 participants, and performs training and verification on 479 participants (945 fundus images) in each of the ASD and TD groups. The participants have an average age of 7.8 years and are mostly boys.

It may be seen that a mean value obtained by calculating gender, full-scale intelligence quotient (FSIQ), and symptom severity depending on a ADOS-2 calibrated severity score, and a mean value obtained by calculating symptom severity depending on the SRS-2 T score were calculated. In the case of TD participants, there are no result values for parameters other than gender in typical development.

The classification model 120 is trained and verified by using the retina image, to which the participant's characteristics is reflected. In the case, mean classification performance and mean calibration performance of the single model and the deep ensemble model that classify ASD and TD are measured to identify a performance difference according to a single model and an ensemble model.

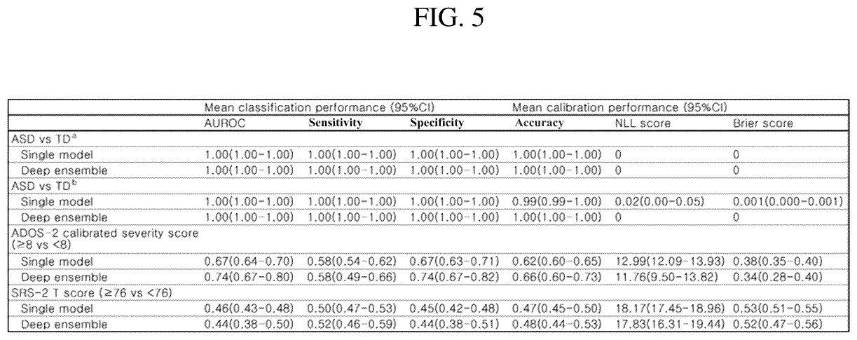

As shown in FIG. 5, it was understood that in a case of screening ASD, the classification model 120 has mean AUROC, sensitivity, and specificity of 1.00 (95% CI, 1.00-1.00) in a test set, and the mean AUROC of 1.00 is maintained even when only 10% of the retina image including an optic nerve head is used.

On the other hand, as shown in FIG. 5, in a case of screening ASD symptom severity, the classification and calibration performance is higher in the ADOS-2 calibrated severity score when the classification model 120 is a deep ensemble model than when the classification model 120 is a single model. In the SRS-2 T score, the classification and calibration performance varies depending on items.

The classification model 120 shows the highest ASD symptom performance when the classification model 120 is a deep ensemble model and is verified by using the ADOS-2 calibrated severity score. In the case, it may be seen that in the test set, the mean AUROC is 0.74 (95% CI, 0.67-0.80), sensitivity is 0.58 (95% CI, 0.49-0.66), and specificity is 0.74 (95% CI, 0.67-0.82).

Accordingly, according to an embodiment of the present disclosure, the classification model 120 does not have a significant difference between a single model and a deep ensemble model in distinguishing between ASD and TD. However, there is a significant difference between a single model and a deep ensemble model in determining ASD symptom severity, and utilizing the deep ensemble model for ASD symptom severity may further improve classification and calibration performance.

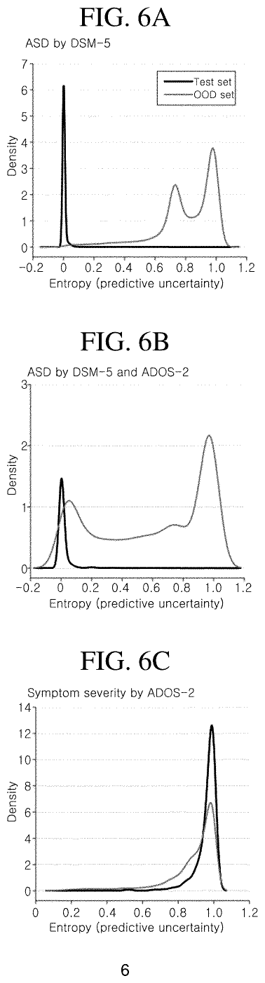

In the meantime, as illustrated in FIGS. 6A to 6C, it is seen that the classification model 120 (e.g. the first model 121) used to screen ASD diagnosed based on the DSM-5 criterion generates significantly low entropy with respect to out-of-distribution sets (OOD) (a). The mean (standard deviation) of the test set is 0.01 (0.03); the mean (standard deviation) of the OOD set is 0.8 (0.2); and, ‘P’ is less than 0.001.

It may be seen that this trend is also observed in the classification model 120 (e.g. the first model 121), which is used to screen ASD by using the DSM-5 criterion and the ADOS-2 scores for the test set and OOD set (b). The mean (standard deviation) of the test set is 0.02 (0.06); the mean (standard deviation) of the OOD set is 0.6 (0.4); and, ‘P’ is less than 0.001.

On the other hand, it may be seen that the classification model 120 (e.g., the second model 122), which is used to screen the ASD symptom severity based on the ADOS-2 score, has high entropy for both the test set and the OOD set. The mean (standard deviation) of the test set is 1.0 (0.06), and the mean (standard deviation) of the OOD set is 0.9 (0.2).

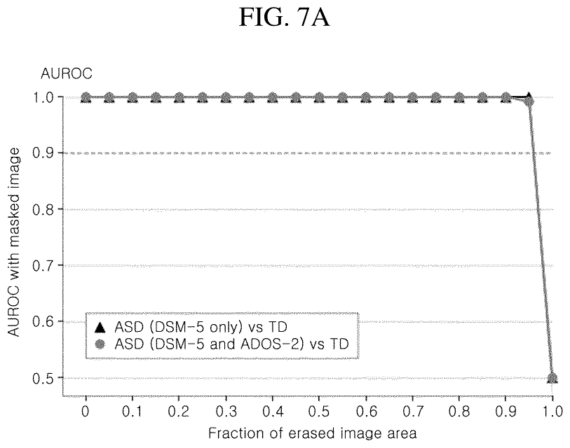

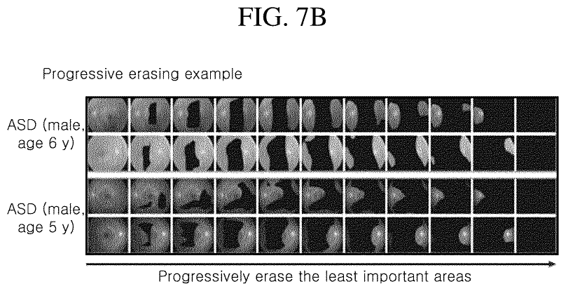

Moreover, as shown in FIG. 7A, it may be seen that the quantitative validation of a heatmap for ASD screening is indicated and a high mean AUROC is maintained even when the fraction of the retina image is removed regardless of an ASD diagnosis method. In other words, in the classification model 120 according to an embodiment of the present disclosure, the mean AUROC is maintained without decreasing, when only the fraction of a retina image of an object or a retina image including an optic nerve head is left and 95% of the least important area is removed. Accordingly, it may be seen that ASD is capable of being screened as long as the retina image includes an optic nerve head area.

Even when only the alpha zone being an area, where red light is emitted in FIG. 7B, and a part of the beta zone around the alpha zone are left, the classification model 120 may maintain high efficiency in consistent mean AUROC by using only the fraction of the retina image.

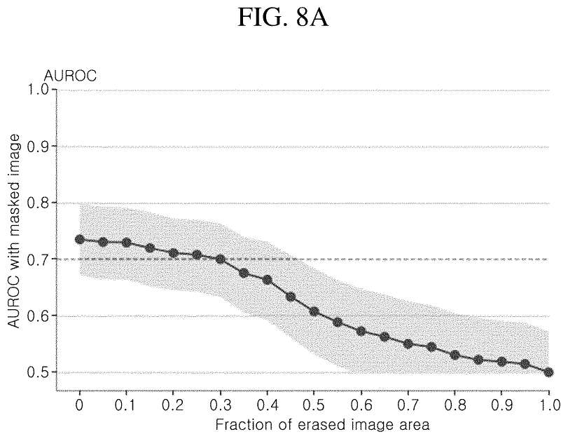

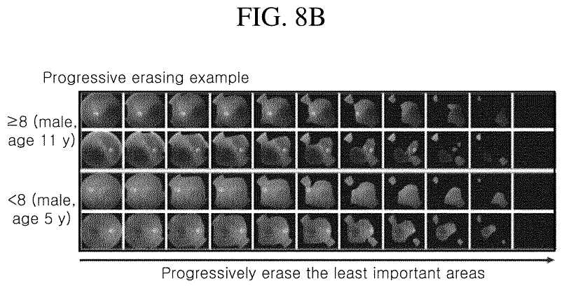

On the other hand, as shown in FIG. 8A, it may be seen that 70% of retina images are required to achieve a mean AUROC of 70% when the ASD symptom severity is screened by using the ADOS-2 score.

As shown in FIG. 8B, a significant amount of the alpha zone and the beta zone in the retina image needs to be left. When only 30% of the retina image is removed and 70% of the retina image is left, the mean AUROC is 0.7. When more than 30% of the retina image is erased, the mean AUROC value may decrease.

Accordingly, the classification model 120 according to an embodiment of the present disclosure may become an objective screening tool for screening ASD by determining whether the ASD is present, and ASD symptom severity by using only the fraction of the retina image or the retina image including the optic nerve head of an object. To maintain the performance of mean AUROC at 70% or higher, the classification model 120 according to an embodiment of the present disclosure may preprocess data such that 70% or higher of the retina image including the optic nerve head is left.

Accordingly, the ASD and the ASD symptom severity may be automatically screened by using it is determined that more than 9 out of 13 retina-related features are significantly different, by using information that the size of the alpha zone and the beta zone where nerve bundles are bundled in the retina image is greater than the TD or that the ratio of the alpha zone and the beta zone to the entire pupil is great. The surrounding area of an ROC area may be detected by setting a high-brightness portion (i.e., a portion of the alpha zone) in the retina image as the ROC area in the data preprocessing process, and thus the mean AUROC performance may be maintained to be high.

In this way, the ASD and ASD symptom severity may be easily evaluated and screened by a person other than a skilled expert by using the electronic device 100 according to an embodiment of the present disclosure.

Meanwhile, the disclosed embodiments may be implemented in a form of a recording medium storing instructions executable by a computer. The instructions may be stored in a form of program codes, and, when executed by a processor, generate a program module to perform operations of the disclosed embodiments. The recording medium may be implemented as a computer-readable recording medium.

The computer-readable recording medium may include all kinds of recording media in which instructions capable of being decoded by a computer are stored. For example, there may be read only memory (ROM), random access memory (RAM), magnetic tape, magnetic disk, flash memory, optical data storage device, and the like.

Disclosed embodiments are described above with reference to the accompanying drawings. One ordinary skilled in the art to which the present disclosure belongs will understand that the present disclosure may be practiced in forms other than the disclosed embodiments without altering the technical ideas or essential features of the present disclosure. The disclosed embodiments are examples and should not be construed as limited thereto.

According to the above-described issues solving means of the present disclosure, the ASD screening process that determines ASD presence and ASD severity by using retina images may be accelerated, thereby solving the issues of limited access to specialized child psychiatric evaluation due to limited resources.

Effects of the present disclosure are not limited to the effects mentioned above, and other effects not mentioned will be clearly understood by those skilled in the art from the following description.

While the present disclosure has been described with reference to embodiments, it will be apparent to those skilled in the art that various changes and modifications may be made without departing from the spirit and scope of the present disclosure. Therefore, it should be understood that the above embodiments are not limiting, but illustrative.

What is claimed is:

1. An electronic device for screening autism spectrum disorder (ASD) and ASD symptom severity based on a retina image, the electronic device comprising:
an input unit configured to receive the retina image;
a classification model configured to classify whether there is the ASD, and the ASD symptom severity based on the retina image by using a deep learning algorithm; and
at least one processor configured to control the input unit and the classification model,
wherein the at least one processor is configured to:
preprocess the received retina image;
train the classification model such that the classification model classifies the ASD and typical development (TD) by using the preprocessed retina image, and classifies the ASD symptom severity; and
allow the trained classification model to screen whether there is the ASD and the ASD symptom severity depending on the input retina image.

2. The electronic device of claim 1, wherein the classification model includes:
a first single model or a first ensemble model configured to determine whether the ASD is present; and
a second ensemble model configured to determine the ASD symptom severity.

3. The electronic device of claim 2, wherein the first ensemble model or the second ensemble model is a deep learning model based on a deep ensemble.

4. The electronic device of claim 3, wherein the classification model is a convolutional neural network that uses ResNeXt-50 network as a backbone.

5. The electronic device of claim 1, wherein the at least one processor is configured to:
classify training or verification data of the classification model into the ASD and the TD depending on only a diagnostic and statistical manual of mental disorders, fifth edition (DSM-5) criterion.

6. The electronic device of claim 5, wherein the at least one processor is configured to:
classify the training or verification data of the classification model into the ASD and the TD depending on the DSM-5 criterion and an ADOS-2 score.

7. The electronic device of claim 6, wherein the at least one processor is configured to:
classify the training or verification data of the classification model depending on the ASD symptom severity based on a score calculated by calculating an ADOS-2 calibrated severity score and an SRS-2 T score.

8. The electronic device of claim 7, wherein the at least one processor is configured to:
perform random undersampling on the retina image before the ASD and the ASD symptom severity are classified; and
perform data segmentation depending on at least one segmentation ratio.

9. The electronic device of claim 8, wherein the at least one processor is configured to:
perform preprocessing of setting and labeling an alpha zone and a beta zone in the retina image as an ROC area before the ASD and the ASD symptom severity are classified.

10. The electronic device of claim 9, wherein the retina image includes information about sizes of the alpha zone and the beta zone, and a ratio of the alpha zone and the beta zone to an entire pupil.

11. A training method for screening ASD and ASD symptom severity based on a retina image as a training method of an electronic device including an input unit configured to receive a retina image and a classification model using a deep learning algorithm, the method comprising:
preprocessing the received retina image;
training the classification model such that the classification model classifies ASD and TD by using the preprocessed retina image, and classifies ASD symptom severity; and
allowing the trained classification model to screen whether there is the ASD and the ASD symptom severity depending on the input retina image.

12. The method of claim 11, wherein the classification model includes:
a first single model or a first ensemble model configured to determine whether the ASD is present; and
a second ensemble model configured to determine the ASD symptom severity.

13. The method of claim 12, wherein the first ensemble model or the second ensemble model is a deep learning model based on a deep ensemble.

14. The method of claim 13, wherein the classification model is a convolutional neural network that uses ResNeXt-50 network as a backbone.

15. The method of claim 11, wherein the training of the classification model such that the classification model classifies the ASD and the TD by using the preprocessed retina image, and classifies the ASD symptom severity includes:
classifying training or verification data of the classification model into the ASD and the TD depending on only a DSM-5 criterion.

16. The method of claim 15, wherein the training of the classification model such that the classification model classifies the ASD and the TD by using the preprocessed retina image, and classifies the ASD symptom severity further includes:
classifying the training or verification data of the classification model into the ASD and the TD depending on the DSM-5 criterion and an ADOS-2 score.

17. The method of claim 16, wherein the training of the classification model such that the classification model classifies the ASD and the TD by using the preprocessed retina image, and classifies the ASD symptom severity further includes:
classifying the training or verification data of the classification model depending on the ASD symptom severity based on a score calculated by calculating an ADOS-2 calibrated severity score and an SRS-2 T score.

18. The method of claim 17, wherein the preprocessing of the received retina image includes:
performing random under sampling on the retina image before the ASD and the ASD symptom severity are classified; and
performing data segmentation depending on at least one segmentation ratio.

19. The method of claim 18, wherein the preprocessing of the received retina image includes:
performing preprocessing of setting and labeling an alpha zone and a beta zone in the retina image as an ROC area before the ASD and the ASD symptom severity are classified.

20. The method of claim 19, wherein the retina image includes information about sizes of the alpha zone and the beta zone, and a ratio of the alpha zone and the beta zone to an entire pupil.
